(19) United States
(12) Patent Application Publication
(10) Pub. No.: US 2016/0353818 A1
(43) Pub. Date: Dec. 8, 2016

(54) NEXT TO SKIN GARMENTS MANUFACTURED FROM A FABRIC COATED WITH A COMPOSITE MATERIAL BASED ON METAL OXIDES AND AINORGANIC MATERIAL

(71) Applicants: GEORGE PSIPSIKAS, ATTICA (GR); ERMIS PSIPSIKAS, ATTICA (GR)

(72) Inventors: GEORGE PSIPSIKAS, ATTICA (GR); ERMIS PSIPSIKAS, ATTICA (GR)

(21) Appl. No.: 14/937,953

(22) Filed: Nov. 11, 2015

(30) Foreign Application Priority Data
Jun. 4, 2015 (GR) 20150100151

(51) Int. Cl. A41D 31/00; D06N 3/14; A41B 1/08; A61F 7/02; A41D 1/06; A41B 11/00; A43B 1/08; A43B 13/10; D06N 3/00; A41D 1/08

(52) U.S. Cl. CPC A41D 31/00; D06N 3/0063; D06N 3/14; D06N 3/0088; D06N 3/0034; A41B 1/08; A41D 1/08; A41D 1/06; A41B 11/00; A43B 1/08; A43B 13/10; A61F 7/02; D06N 2211/10; D10B 2331/02; D10B 2501/06; A61F 2007/0045

(57) ABSTRACT

A non compression garment and specially “next to skin” articles based on a fabric coated with a composite material on the basis of metal oxides and inorganic material emitting in the far infrared range which comprises a composite material including a polymer matrix with embedded particles of an inorganic material capable of emitting far-infrared radiation, a fabric surrounding a body part of the user and having at least a part or the whole of it covered with the said composite material which comprises metal oxides and inorganic material, where the fabric is printed with the composite material for forming a pattern, and surrounds any body part of the user, such as arms, throat, torso, thigh, or calf for increasing of the temperature of the skin, improving blood circulation to the microcapillaries thereunder, to the upper and lower limbs, improving the muscle power, decreasing the lactic acid in the muscles and the free radicals, and for reducing the appearance of cellulite.

### FIELD OF THE INVENTION

The invention relates to non-compression clothing items, which cover a part or the whole of the human body and come in contact with the skin, characterized in that they are manufactured from a fabric covered with a composite material which is based on metal oxides and an inorganic material, which emit radiation in the far infrared.

The far-infrared radiation, in particular in a wavelength range of 4 microns to 14 microns length, has the advantage of absorbing the infrared rays emitted by the body and then reflecting them back to it, thereby heating the body with beneficial health results. Garments of this design may be used both in the context of sports and in daily activities to improve the comfort and health of the user.

The high level of emissivity in the wavelengths of the far infrared of the invention increases the temperature at the surface of the skin. This implies an increase in the circulation of the flow of red corpuscles in the micro capillaries under the skin, which according to the medical literature leads to improved oxygen exchange in the muscle cells and thus to faster recovery after exercise and constant muscle power, reducing the lactic acid build-up in muscles and thus reducing pain. Furthermore it ensures a better elimination of toxins and free radicals from within the muscles and thus helps to reduce the appearance of cellulite.

### STATE OF THE ART

Until now, so-called “compression” garments are known, which are configured to apply an elastic force around a part of the body of a user. The produced compression generates physiological effects on the user by the application of compression on the skin. The compression levels are defined in various official standards (e.g. AFNOR standards in France or GZG standards in Germany).

The said garments have the main disadvantage that they are difficult to wear, while due to the pressure they apply on the skin there are not comfortable.

Also, various inorganic materials are known, which have the property of absorbing energy radiation for re-emission mainly in the far infrared, and different types of clothing have been developed by combining a fibrous tissue with a resin including these materials.

The fibers are produced by including powders of inorganic materials within a resin, and then producing a filament from this mixture. The filament is then woven to weave the fabric.

However, the presence of the powder inside the fibers greatly reduces their elasticity and tensile strength. Therefore, these fibers have a high rate of wear and a reduced life span. Therefore, the specific fibers are unsuitable for garments, which have to show significant elasticity or in case of clothing for sports, where because of high friction an increased wear is caused.

Also anti-cellulite garments are known using a fabric comprising a composite of a polymeric material and an inorganic material in form of fibers. The said garments do not give enough satisfaction in term of emissivity of far infrared radiations. Far infrared emissive radiations is a surface property, in this case external surface of the fibbers. The average level of emissivity of this kind of fibbers is empirically zero and is around 70 to 80% of the emissivity in far infrared radiation.

Also a far infrared emitting fabric is known, which constitutes a compact film laminate on a fabric. Specifically, the fabric is coated a resin, a catalyst and inorganic materials emitting in far-infrared are mixed in to form a well-defined polymer blend proportion. The polymer mixture is applied to a release paper and dryed to from a film. The film is then applied to a fabric or knitted fabric with the help of an adhesive. The fabric is then cut according to different patterns to make clothes. The disadvantage of the said fabrics is that the film tends to wear very fast and also sweat removal is difficult.

Also garments are known by means of which muscle power is increased and lactic acid is reduced. The said garments are based on the physical property of muscle compression on specific parts of the body, providing better maintenance of this effect and allowing a better return of blood flow.

The fibers used to make such compression garments are a blend of elastic fiber threads like elastane and classic fibber threads based on polyamide or polyester, which are mechanically blended and knitted to form fabric from which a final garment is formed. The said garments are difficult to put on and often not very comfortable.

Finally garments are known, by means of which blood flow in micro capillaries directly under the skin is increased, which help to reduce free radicals. In practice, the fabric used to make these garments is based on a mix of fibers and specially electrical conductive fibers attached to a battery for heating up the fabric. Increase of the temperature of the human could help to increase the blood flow in the micro capillaries under the skin witch helps to reduce free radicals. The disadvantage of this kind of garments made from a mix of fibbers and especially electrical conductive fibers is that a battery is required for their recharging. Also they are not usable since the fabric has no elasticity.

The said disadvantages urged us to find a solution, the result of which is the object of the present description, since our invention eliminates the majority of the above disadvantages due to the fact that the proposed product is not a compression garment.

### OBJECTS OF THE INVENTION

Object of the present invention is to solve the problems of the prior state of the art, by providing a high level of emissivity in the far infra red rays of the fabric coated with composite material, instead of a fabric produced with fibbers with powder of inorganic material and resin, in that it uses at least one coated layer whose function is insulating the user's body and emitting towards the body also infrared rays that escape through the above of the high far infrared emittive coated fabric.

Another object of the present invention is to provide a better air permeability of the fabric against the film as well as better comfort of the garment when worn by the user.

When using the object of the invention, an improved blood flow in the micro capillaries under the skin without muscle compression is established, since in practice, too much compression causes an unpleasant sensation during sporting or discomfort in the movements

Finally due to the increase of the temperature of the user's body, an increase of body performance, a reduction of lactic acid in muscles, a tendency to reduce free radicals in muscles and a reduction of cellulite are achieved.

### BRIEF DESCRIPTION OF THE INVENTION

The invention regards non compression garments which cover the whole or part of the human body, and come in contact with the skin, which may be T-shirts, pairs of shorts, trousers, leggings, socks and shoe soles and/or shoes, under-bedsheets, characterized in that they come in contact with the skin, based on a fabric coated with a composite material on the basis of metal oxides and an inorganic material emitting in the far infrared rays range by means of which the physical performance, and blood flow in micro capillaries under the skin, are increased resulting to a reduction of free radicals and reduction of the appearance of cellulite,

characterized in that they comprise:
A far infra red emitting composite with an emissivity level of more than 95% between 4 and 14 microns.
A far infrared emitting composite including a polymer matrix in which are embedded particles of metal oxides and inorganic material capable of emitting far-infrared radiation;
A fabric intended to surround a body part of the user and having at least a part coated with said far infrared composite.

The far infrared composite is applied by impregnation or by any other suitable way. Also, the composite material designs are spaced from each other and they may be spaced apart from each other in all directions in the plane of the fabric by a distance of at least 1.5 mm and may have less than 2 cm².

The garments incorporating the features of the present invention may be T-shirts with long or short sleeves, trousers, pair of shorts, leggings, underwear, bathing suits, stockings, socks, gloves, unter-bedsheets and may cover the human body partly or totally.

The metal oxides and inorganic material part used in the composite material of the invention has the form of a powder capable of emitting far-infrared radiation. Said powder may contain at least one oxide of the following: Al₂O₃, Fe₂O₃, TIO₂, Cr₂O₃, SiO₂, MgO, ZrO₂, MnO₂, CO₂O₃, and inorganic material having a radiation peak at a wavelength between 4 and 14 microns.

The polymer matrix used in the composite material of the invention is in a form based on water or solvent, capable to surround and to mix properly with the powder of metal oxides and mineral material.

Also the invention also relates to a method of manufacturing the fabric from which the said garments are manufactured , comprising
Coating a fabric with a composite material including a polymer matrix in which particles of an inorganic material capable of emitting far-infrared radiation are embedded;
Transforming the fabric to form the garment, said processing assembly comprising two fabric edges.

The technical features and advantages of the present invention will become apparent from the following description.

### DETAILED DESCRIPTION OF THE INVENTION

For the purposes of the present invention, far infrared radiation is infrared radiation the wavelength of which is for example between 2 microns and 200 microns. Advantageously, it is infrared with a wavelength between 4 and 14 microns and provides a high level of emissivity of more than 95% in the range of 4 to 14 microns.

The invention regards a non compression garment comprising a composite material including a polymer matrix in which particles of an inorganic material are embedded. This inorganic material is capable of transmitting and reflecting far infrared radiation. The garment comprises a fabric coated with at least one part of said composite material surrounding the user.

The invention provides the physiological effects of far infrared radiation, such as an increase in the temperature of the skin, increase in blood flow in the micro capillaries under the skin by which the free radicals and muscle pain may be reduced, and respectively muscle power may be increased and cellulite be reduced.

The garment comprises a fabric for covering around a portion of the defined body of the user. The fabric can thus be adapted to surround a member of the user, such as the arms, throat, torso, thigh or calf. The fabric has at least a portion of the composite material coated with a surface. The composite material includes a polymer matrix in which in particles of an inorganic material capable of emitting far-infrared radiation are embedded or incorporated.

In particular, the composite material may comprise a matrix made of a breathable hydrophilic polymer, but impermeable to liquid water and, preferably, windproof. Such properties are particularly advantageous for a garment for a sport. The matrix may be based on polytetrafluoroethylene (PTFE), polyurethane, polyester (PE), acrylic, polyvinyl chloride (PVC), polystyrene-butadiene copolymer (SBS), or an artificial latex (polyisoprene), or any other thermoplastic or thermosetting polymers.

The composite material also contains mineral oxides and inorganic material in the form of particles of powder emitting infrared radiation (the term far-infrared designates hereafter material reemitting a significant part of the radiation or energy received as radiation with a wavelength between 4 and 200 microns, preferably between 4 and 14 microns) distributed, preferably homogeneously, in the polymer matrix. More specifically, the inorganic material may present a peak radiation at a wavelength between 4 and 14 microns.

The inorganic material are selected from mineral oxides and inorganic material of the following list: Al₂0₃, Fe₂O₃, TiO₂, Cr₂0₃, Si0₂, MgO, Zr0₂, MnOy, C02O3, Y2O3.Also, mixtures of the above oxides or natural minerals may be used.

Far infrared can increase skin temperature and cause a sensation of heat when absorbed by the skin (epidermis and/or dermis) by converting the radiation into heat. Also it increases blood flow to the skin through the dilation of capillaries and should reduce free radicals. Oxygenation is improved which increases muscular performance. Far infrared further activates the metabolism and reduces the sensation of pain and the risk of injury and reduces the appearance of cellulite. Also the recovery procedure is facilitated.

The fabric is advantageously realized in a conventional material having a high elasticity. This material may comprise for example Elastane, a non micro fibre polyamide (for its aesthetic properties and transparency) polyamide micro fibres (for its properties of elasticity, comfort, softness and resistance), cotton (especially for sensitive skin and its limiting properties of sweating), natural rubber (for its properties of elasticity and low environmental impact) or micro wool (for opacity, absorbency, its softness, its insulating properties and its low environmental impact). The type of fabric used can be variable. For example, the fabric can be selected from the non-woven fabrics, woven fabrics (woven fabric), knit fabrics (mesh) and their combinations. In any case, the fabric should have good elasticity and stretch.

The preparation of the fabric takes place either by a direct covering/printing procedure or by a dipping procedure or by tumblering

In the first case, for the preparation of the fabric, a mixture of elastic fibers is used such as Elastane and the procedure of direct covering/printing is used, where the metal oxides and the inorganic material are used as follows:

| Oxide | % By weight |
|---|---|
| TiO₂ | 30-40% |
| SiO₂ | 10-20% |
| MnO₂ | 10-20% |
| Al₂O₃ | 10-20% |
| Fe₂O₃ | 5 to 10% |
| MgO | 3-7% |
| Co₂O₃ | 3-7% |
| ZrO₂ | ≦5% |

Alternatively, the same mixture of elastic fibers may be used as a base fabric, however the covering may be formed from a slurry produced from hydrophilic polyurethane, which is in the form of a dispersion in an aqueous phase.

In this case, slurry paste contains the following ingredients:
Aliphatic ether ester polyurethane hydrophilic dispersion in water in proportion 50:50 by weight of the dispersion, and 80% by weight. In this case, water is the carrier liquid.
Mineral oxydes dispersed in the slurry, 15% by weight
crosslinking agent: 5% by weight

It is also possible to add pigments and/or dyes in the composition.

The slurry is coated or printed on the fabric, using an application head. The assembly is then dried at about 100° C., and then subjected to a temperature of about 160° C. to crosslink the polymer matrix, in a drying oven for curing. The composite material thus obtained contains 30% by weight of ceramic and 70% by weight of polymer matrix. The fabric material is then confectioned in a garment.

During dipping procedure, the formulation is formed from a slurry made of a polyurethane and mineral oxides diluted in water to reduce the viscosity for a better absorption during the process.

The slurry contains the following components:

Bulk A is composed by polyurethane, mineral oxides and crosslinker with the following formulation
polyurethane dispersion in water in proportion 50:50 by weight of the dispersion, and 80% by weight. Water is the carrier liquid.
Mineral oxydes dispersed in the slurry, at 15% by weight
crosslinking agent, at 5% by weight

Bulk A well mixed with water in a ratio of 50% of each and bulk B is formed.

It is also possible to add pigments and/or dyes in the composition.

Bulk B is applied by dipping on connective fabric placed in a tray, passing the fabric in the bath. the fabric is output from the tray and passes between two rollers to express the excess of bulk B, then passes through an oven at 100° C. to dry and then at 160 ° C. to polymerize.

The fabric material is then confectioned in a garment.

During preparation of the garment by tumblering, a fabric made of a mixture of Elastane and polyamide is used. For the tumblering process the formulation is formed from a slurry made of a polyurethane and mineral oxides diluted in water to reduce the viscosity for a better absorption during the process, with components similar to those used during dipping process.

In the specific procedure, the garments are placed in the drum, where the Bulk B is added in the proper proportions, so that Bulk B has a homogeneous concentration on each garment.

This process is effected a temperature ca. 100° C. to accelerate the evaporation of water.

When the process comes to an end, the clothes are now homogeneously impregnated with bulk B, and tt is sufficient to treat at 160° C. to complete the polymerization of the Bulk B.

In order to confirm the advantageous effect of the invention, an experimental study was performed, in which twenty four healthy volunteers, 23 to 77 years of age, 13 men and 10 women, participated. In the pilot study, the volunteers wore a short-sleeved T-shirt based on a fabric printed with a composite material according to the present invention, for various time periods ranging from 2 hours per day for 6 days to 8 hours per days for 30 consecutive days.

Optimal results were observed with those volunteers that had used the garment for 2 hours per day for 6 days.

Based on these conditions (2 hours per day for 6 days), the levels of free radicals in PBMC were determined for these subjects.

Specifically, it was observed that 15 subjects, i.e. 62,5% of all participants, showed decreased levels (58.7% on average, total decrease range from 9.13% to 95.38%) of free radical species in PBMCs after the use of composite material—non-compression garment.

The following tables demonstrates the results after the use of the garment (T-shirt) incorporating the present invention.

TABLE
Free radical levels (ROS) in PBMCs before and after the results

| | | Free radical levels (ROS) | |
|---|---|---|---|
| | | Subjects with reduced levels after | % reduction rate |
| Volunteers | | the use of a short-sleeved T-shirt (%) | (range) |
| Total | 24 | 62.5 (15/24) | 9.13-95.38 |
| men | 14 | 71.4 (10/14) | 30.14-95.38 |
| women | 10 | 50.0 (5/10) | 9.13-77.54 |

It should be noted that the present invention is not limited to the above exemplary embodiments but it may be applied with any kind of clothing and footwear or associated accessory coming in contact with the skin

1. A non-compression garment wherein the garment comprises:
a composite material including a polymer matrix in which particles of an inorganic material capable of emitting far-infrared radiation are embedded; and
a fabric for surrounding a body part of a user and having at least a portion coated with said composite material.

2. The non-compression garment according to claim 1, wherein the composite material contains metal oxides and inorganic material and at least one of the following components Al₂O₃, Fe₂O₃, TiO₂, Cr₂O₃, SiO₂, MgO, ZrO₂, MnO₂, or Co₂O₃.

3. The non-compression garment according to claim 1, wherein the composition of the metal oxide and inorganic material has the following composition by weight, the percentage being 100%

| | |
|---|---|
| TiO₂ | 30-40% |
| SiO₂ | 10-20% |
| MnO₂ | 10-20% |
| Al₂O₃ | 10-20% |
| Fe₂O₃ | 5 to 10% |
| MgO | 3-7% |
| Co₂O₃ | 3-7% |
| ZrO₂ | ≦5%. |

4. The non-compression garment according to claim 1, wherein the composite material contains a polymer matrix based on a breathable hydrophilic polymer.

5. The non-compression garment according to claim 1, wherein the polymer matrix is based on a water based polyurethane.

6. The non-compression garment according to claim 1, wherein the fabric is selected from a fabric or a combination of fabric made from threads, fibers or other suitable natural or synthetic material.

7. The non-compression garment according to claim 1, wherein the garment comprises a fabric and a composite material where the fabric is printed with the composite material to form a pattern and surround a body part of the user in order to increase the temperature of the skin, improve blood circulation in the microcapillaries under the skin, improve the muscle strength, reduce the lactic acid in the muscles and the free radicals, or to reduce the appearance of cellulite.

8. The non-compression garment according to claim 1, wherein the fabric is impregnated with the composite material by a dipping process.

9. The non-compression garment according to any preceding claim 1, wherein the fabric is impregnated with the composite material by a tumblering process.

10. The non-compression garment according to claim 1, wherein the composite material has a level of emissivity of at least 95% in the far infrared radiation between 4 and 14 microns.

11. The non-compression garment according to claim 4, wherein the hydrophilic polymer is polytetrafluoroethylene (PTFE), polyurethane, polyester (PE), acrylic, polyvinyl chloride (PVC), polystyrene-butadiene copolymer (SBS), or an artificial latex (polyisoprene), or other thermoplastic or thermosetting polymer.
